(19) United States
(12) Patent Application Publication
(10) Pub. No.: US 2018/0056023 A1
(43) Pub. Date: Mar. 1, 2018

(54) Moisture Retaining Mask

(71) Applicant: Shunsheng Han, Los Alamos, NM (US)

(72) Inventor: Shunsheng Han, Los Alamos, NM (US)

(21) Appl. No.: 15/462,884

(22) Filed: Mar. 19, 2017

Related U.S. Application Data
(60) Provisional application No. 62/381,397, filed on Aug. 30, 2016.

(51) Int. Cl. A61M 16/16; A61M 16/06

(52) U.S. Cl. CPC A61M 16/16; A61M 16/0683

(57) ABSTRACT

A moisture retaining mask configured to be fitted over a mouth of a user as the user sleeps to add water containing moisture to the breath of the user while the user is inhaling, the mask including a main portion having an outer layer, an inner layer, and a middle layer, the main portion configured to extend to substantially cover at least the mouth of the user; the middle layer comprising a water absorbing material; and, a wick configured to convey a water containing fluid, the wick extending from a reservoir holding the water containing fluid to the middle layer, the reservoir disposed proximate the main portion.

### TECHNICAL FIELD

This Application claims the benefit of provisional application 62/381,397 filed 30 Aug. 2016. The present disclosure relates generally to a moisture retaining mask. More particularly, the present disclosure relates to a moisture retaining mask for retaining moisture in a congested user's airway during sleep.

### BACKGROUND

At some point or another, all people are affected by some other illness that causes them to be congested, such as the common cold, the flu, or allergies. In fact, more than 60 million people currently suffer from allergies, making this a widespread problem. While during the day congestion is merely a nuisance, when a congested person attempts to sleep, their congestion can greatly diminish the quality of sleep obtained, and can prevent them from sleeping all together.

Chief among the problems with sleep caused by congestion is the drying out of the mouth, throat, trachea, and bronchi. This is caused by a person having to leave their mouth open during sleep, since that person's sinuses are too clogged to allow air to pass through. This dryness can cause a congested person to stay awake all night; and can cause lasting damage to the person's mouth, throat, trachea, and bronchi.

While medications do exist that allows congested people to sleep through the night despite their dry mouth, these medications leave the user waking up with an incredibly dry mouth and throat, which causes the user considerable discomfort throughout the day. Over time, this dryness compounds and can result in severe problems. As such, there exists a need for a device that can provide a congested user with quality sleep, all while retaining moisture in the user's mouth so that they can get a good night's sleep and not wake up with a dry mouth, throat, trachea, and bronchi.

While these units may be suitable for the particular purpose employed, or for general use, they would not be as suitable for the purposes of the present disclosure as disclosed hereafter.

In the present disclosure, where a document, act or item of knowledge is referred to or discussed, this reference or discussion is not an admission that the document, act or item of knowledge or any combination thereof was at the priority date, publicly available, known to the public, part of common general knowledge or otherwise constitutes prior art under the applicable statutory provisions; or is known to be relevant to an attempt to solve any problem with which the present disclosure is concerned.

While certain aspects of conventional technologies have been discussed to facilitate the present disclosure, no technical aspects are disclaimed and it is contemplated that the claims may encompass one or more of the conventional technical aspects discussed herein.

### BRIEF SUMMARY

An aspect of an example embodiment in the present disclosure is to provide a means for keeping a congested user's airway moist during sleep. Accordingly, the present disclosure provides a moisture retaining mask for adding moisture to their air as it is inhaled through the open mouth of a congested user, preferably while that user is asleep.

Accordingly, the present disclosure describes a moisture retaining mask for use by a user by placing over the user's mouth as the user sleeps, adding moisture to the breath of the user. The moisture retaining mask includes a main portion which is constructed out of an inner layer, a middle layer, and an outer layer. The inner and outer layers are constructed out of moisture-resistant material, while the middle layer is constructed out of a moisture-absorbing material. The main portion also has a border region where the inner and outer layers are secured to each other. The middle layer has a pair of plys that extend parallel to each other. The moisture retaining mask also includes a water reservoir, for holding a quantity of water, which is attached to a bottom end of the main portion. The moisture retaining mask also includes a fluid conveying mechanism for carrying water from the reservoir to the middle layer. This fluid conveying mechanism includes a wick for absorbing and wicking water from the reservoir to the middle layer. This wick has a pair of ends; one end terminating in the reservoir and the other end terminating between the plys of the middle layer. The fluid conveying mechanism also has a tube that extends partially into the reservoir and partially into the middle layer. The wick extends fully through this tube, and the tube provides a liquid barrier that prevents moisture from escaping from the wick as it travels between the reservoir and the middle layer. Further, the moisture retaining mask includes at least one strap attached to the main portion for securing the water retaining mask to a user's head. In some embodiments, the water retaining mask includes an opening in the outer layer proximate to the water reservoir. This opening has a front tube that extends from the water reservoir through the opening, allowing additional moisture from the air to enter into the reservoir. In other embodiments, the front tube has a clamp that is capable of partially or completely closing said front tube.

The present disclosure addresses at least one of the foregoing disadvantages. However, it is contemplated that the present disclosure may prove useful in addressing other problems and deficiencies in a number of technical areas. Therefore, the claims should not necessarily be construed as limited to addressing any of the particular problems or deficiencies discussed hereinabove. To the accomplishment of the above, this disclosure may be embodied in the form illustrated in the accompanying drawings. Attention is called to the fact, however, that the drawings are illustrative only. Variations are contemplated as being part of the disclosure.

### BRIEF DESCRIPTION OF THE DRAWINGS

In the drawings, like elements are depicted by like reference numerals. The drawings are briefly described as follows.

FIG. 1A is a diagrammatic perspective view, showing an example embodiment of the moisture retaining mask according to the present disclosure.

FIG. 1B is a diagrammatic perspective view, showing an alternative example embodiment of the moisture retaining mask according to the present disclosure.

FIG. 2 is a front view, of just the mask, per se, according to the present disclosure.

FIG. 3 is a front elevational view with parts broken away, showing the internal components of an example embodiment of the moisture retaining mask according to the present disclosure.

FIG. 4 is a cross-sectional side view of an example embodiment of the moisture retaining mask according to the present disclosure.

The present disclosure now will be described more fully hereinafter with reference to the accompanying drawings, which show various example embodiments. However, the present disclosure may be embodied in many different forms and should not be construed as limited to the example embodiments set forth herein. Rather, these example embodiments are provided so that the present disclosure is thorough, complete and fully conveys the scope of the present disclosure to those skilled in the art.

### DETAILED DESCRIPTION OF THE PREFERRED EMBODIMENTS

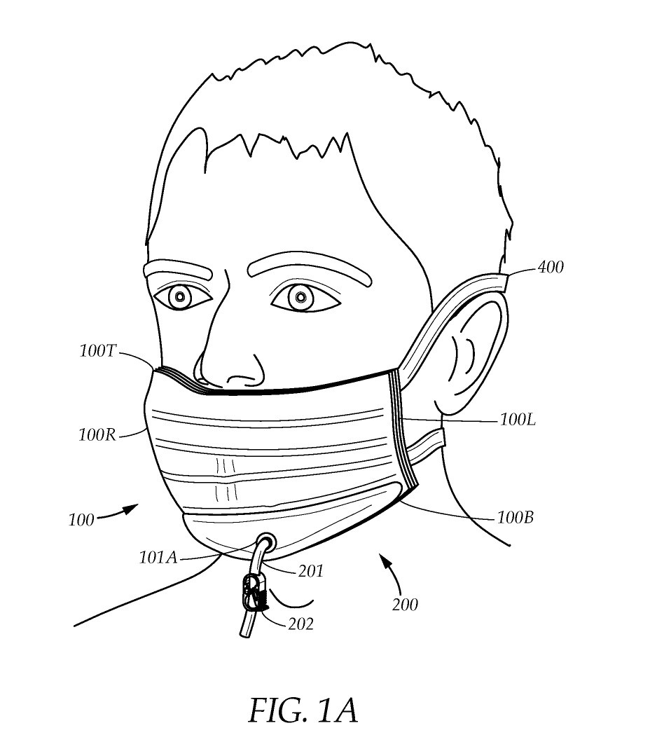

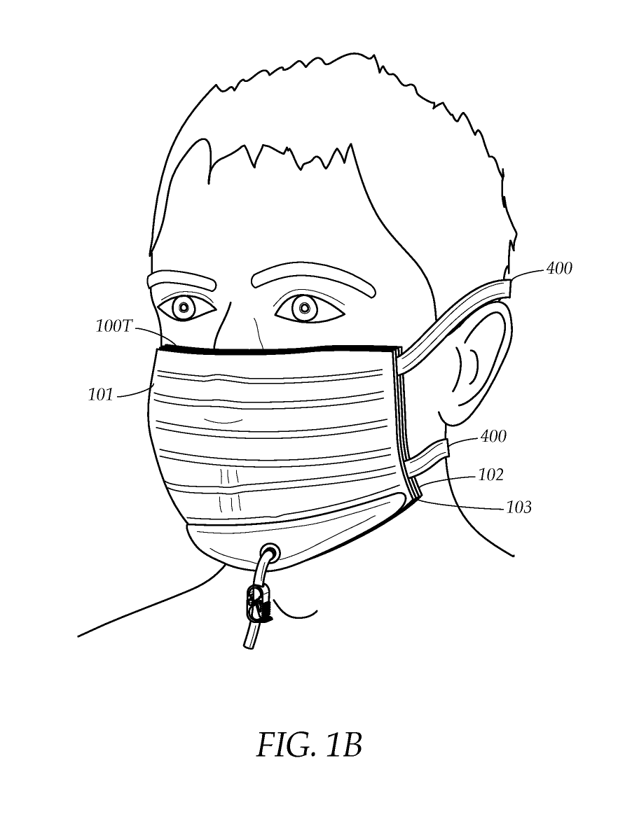

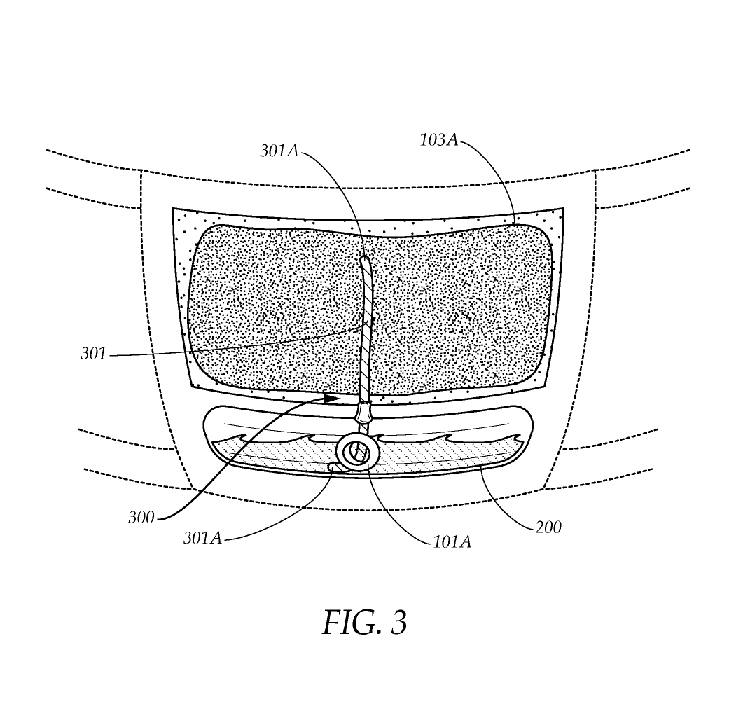

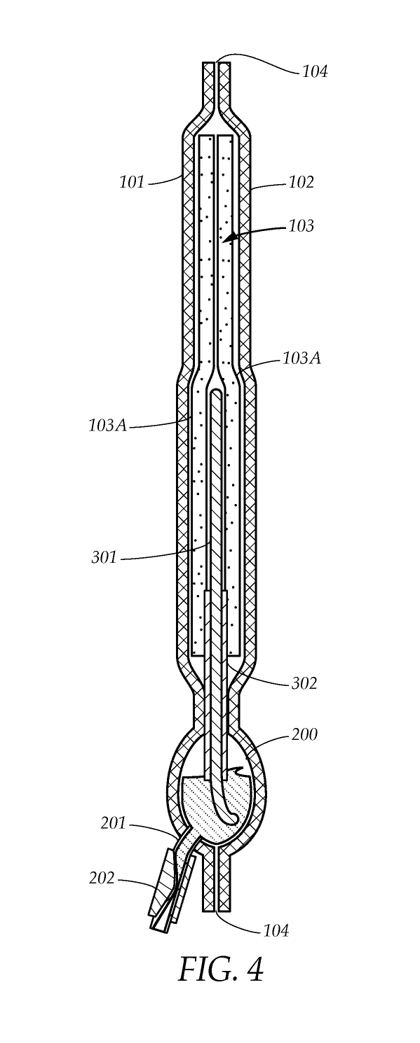

FIG. 1A illustrates a moisture retaining mask having a main portion 100, a water reservoir 200, a fluid conveying mechanism 300 (see FIG. 3), and at least one strap 400. The moisture retaining mask is to be used by a user with a mouth and is for placing over said mouth as the user sleeps, such that it will add moisture to the breath of the user. The main portion 100 has a top side 100T, a bottom side 100B, a left side 100L, a right side 100R, and has an outer layer 101, an inner layer 102, and a middle layer 103. The outer layer 101 and the inner later 102 are constructed out of a moisture resistant material and the middle layer 103, which includes a pair of plys 103A (see FIG. 4) and is constructed out of a moisture absorbing material. Preferably, said moisture resistant material will be a breathable fabric, such as polyester. Preferably, the moisture absorbing material will be a fabric such as a cotton towel. The use of flexible materials provides for a limited amount of dead space in between the inner layer 102 and the mouth of the user. Further, because the water reservoir 200 is located beneath the main part 100, a user wearing the moisture retaining mask will be able to sleep on their side without interrupting the function of the moisture retaining mask.

In one preferred embodiment, the outer layer 101 includes front opening 101A. In this same preferred embodiment, the water reservoir 200 is integrated with a front tube 201, which extends through the front opening 101A and the outer layer 101 such that it protrudes from the moisture retaining mask. Said front tube 201 provides a mechanism for allowing water to fill the water reservoir 200. In another preferred embodiment, the front tube 201 has a clamp 202 attached to it. The clamp 202 allows the user to selectively open the front tube 201 for filling the reservoir, and close the front tube 201 to prevent leakage therefrom. This in turn will allow the user to control the amount of moisture that flows through the fluid conveying mechanism 300, as described hereinafter. Referring to FIG. 1B, an alternative embodiment of the moisture retaining mask is shown. Here, the top surface 100T extends above a nose of the user when in use.

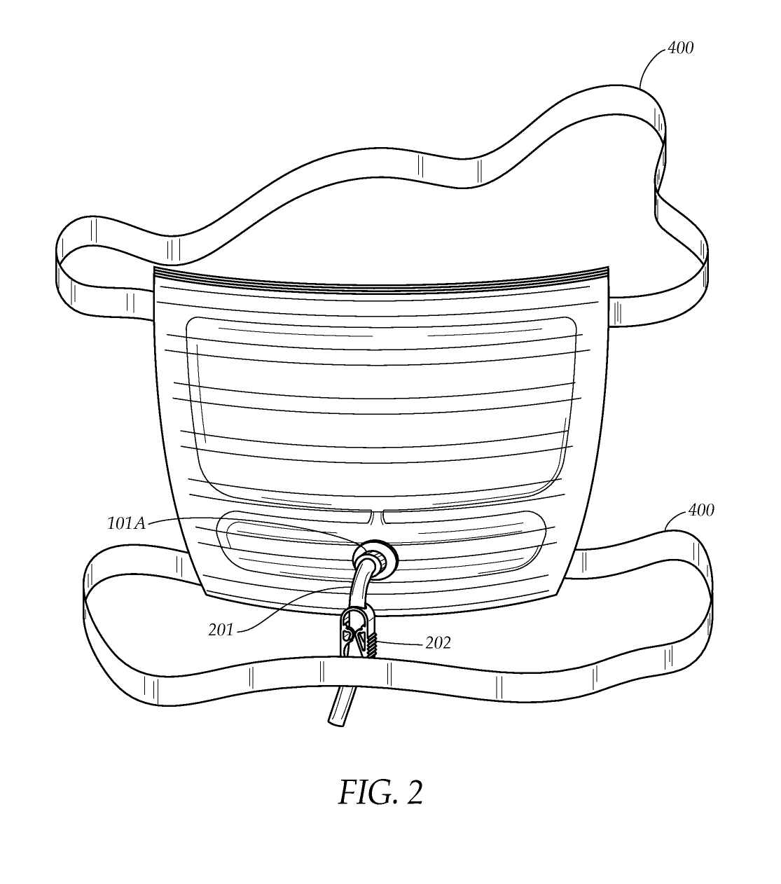

FIG. 2 shows a front view of the moisture retaining mask, where the front opening 101A, the front tube 201, the clamp 202, and the at least one strap 400 are shown in greater detail. Here, a preferable embodiment of the clamp 202 is shown, where said clamp 202 is a tubing clamp, as sold by the Halkey Roberts Corporation of Saint Petersburg, Florida. Other types of clamps are suitable for use as the clamp 202. Further, in this embodiment, two of the at least two straps 400 are shown attached to the main portion 100. Having two of the at least two straps 400 provides optimized support of the moisture retaining mask when worn by the user. Preferably, the at least two straps 400 are adjustable to provide optimal fit for the user wearing the moisture retaining mask.

Referring to FIG. 3, the internal structure of an embodiment of the moisture retaining mask is shown. In particular the water reservoir 200 and a fluid conveying mechanism 300 is shown, where the fluid conveying mechanism 300 features a wick 301, having a pair of ends 301A, and a tube 302. Also shown is the front opening 101A. Here, a volume of water is shown within the water reservoir 200. Any type of potable water is suitable for use in the moisture retaining mask of the present disclosure. In a highly preferred embodiment, 100 mL of water is disposed in the water reservoir 200. The water contained in water reservoir 200 gets absorbed by the wick 301 and one end of the pair of ends 301A and gets passively conveyed through said wick 301 to the other end of the pair of ends 301A. As can be seen, a portion of the wick 301 and one of the ends of the pair of ends 301A are within the pair of plys 103A. As the middle layer 103 and the pair of plys 103A are constructed out of a moisture absorbent material, they will absorb any fluid conveyed by the wick 301. An optional flow control mechanism, such as a clamp, can be added to tube 302 to adjust the speed of water wicked into the middle layer 103. Additional flow control can be created by placing knots in the wick 301 itself.

FIG. 4 shows a side view of the moisture retaining mask. Here, the moisture retaining mask includes the outer layer 101, the inner layer 102, the middle layer 103 including the pair of plys 103A, a border region 104, the wick 301, a tube 302, the water reservoir 200, the front tube 201, and the clamp 202. As can be seen, the outer layer 101 and the inner layer 102 are secured to each other at the border region 104. The pair of plys 103A are disposed between the inner layer 102 and the outer layer 101. Here, the wick 301 extends between the water reservoir 200 and the middle layer 103, and is disposed between the pair of plys 103A. Partially surrounding the wick 301 is the tube 302. This limits the amount of moisture that is conveyed by the wick but is not conveyed to the pair of plys 103A. The tube 302 is constructed out of a liquid-impermeable material to further improve the efficiency of conveying by the wick 301. The front tube 201 extends from the water reservoir 200, through the outer surface 101.

The moisture retaining mask of the present disclosure operates by moistening the air inhaled by the user of the mask by having the air pass through the middle layer 103, which has been moistened by fluid conveying mechanism 300. When a user exhales, the moisture in the exhaled air will condense on moisture retaining mask as it is relatively cold compared to the user's breath. Beneficially, the heat from the exhaled air will, due to the moisture retaining mask, be partially transferred to the air subsequently inhaled by the user. Note that the examples provided of the mask are non-limiting, and may be varied significantly while adhering to the spirit of the disclosure.

It is understood that when an element is referred hereinabove as being “on” another element, it can be directly on the other element or intervening elements may be present therebetween. In contrast, when an element is referred to as being “directly on” another element, there are no intervening elements present.

Moreover, any components or materials can be formed from a same, structurally continuous piece or separately fabricated and connected.

It is further understood that, although ordinal terms, such as, “first,” “second,” “third,” are used herein to describe various elements, components, regions, layers and/or sections, these elements, components, regions, layers and/or sections should not be limited by these terms. These terms are only used to distinguish one element, component, region, layer or section from another element, component, region, layer or section. Thus, “a first element,” “component,” “region,” “layer” or “section” discussed below could be termed a second element, component, region, layer or section without departing from the teachings herein.

Spatially relative terms, such as “beneath,” “below,” “lower,” “above,” “upper” and the like, are used herein for ease of description to describe one element or feature's relationship to another element(s) or feature(s) as illustrated in the figures. It is understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device can be otherwise oriented (rotated 90 degrees or at other orientations) and the spatially relative descriptors used herein interpreted accordingly.

Example embodiments are described herein with reference to cross section illustrations that are schematic illustrations of idealized embodiments. As such, variations from the shapes of the illustrations as a result, for example, of manufacturing techniques and/or tolerances, are to be expected. Thus, example embodiments described herein should not be construed as limited to the particular shapes of regions as illustrated herein, but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

In conclusion, herein is presented a moisture retaining mask. The disclosure is illustrated by example in the drawing figures, and throughout the written description. It should be understood that numerous variations are possible, while adhering to the inventive concept. Such variations are contemplated as being a part of the present disclosure.

What is claimed is:

1. A moisture retaining mask configured to be fitted over a mouth of a user to add water containing moisture to the breath of the user while the user is inhaling, comprising:
a main portion having an outer layer, an inner layer, and a middle layer, the main portion configured to extend to substantially cover at least the mouth of the user;
the middle layer comprising a water absorbing material; and,
a wick configured to convey a water containing fluid, the wick extending from a reservoir holding the water containing fluid to the middle layer, the reservoir disposed proximate the main portion.

2. The moisture retaining mask of claim 1, further comprising a top, bottom, left side and right side, the outer layer and inner layer spanning substantially between the top, bottom, left side and right side, and having a border region wherein the outer layer is secured to the inner layer, the middle layer spanning nearly to the top, bottom, left side and right side, up to the border region.

3. The moisture retaining mask of claim 1, the outer layer and inner layer comprising a water resistant material.

4. The moisture retaining mask of claim 3, the water resistant material comprising polyester.

5. The moisture retaining mask of claim 1, the water absorbing material comprising cotton.

6. The moisture retaining mask of claim 1, the middle layer comprising a pair of plys that extend parallel to each other.

7. The moisture retaining mask of claim 6, one end of the wick disposed between the plys.

8. The moisture retaining mask of claim 6, the pair of plys comprising a water absorbing material.

9. The moisture retaining mask of claim 1, at least the main portion comprising flexible materials.

10. The moisture retaining mask of claim 1, the wick at least partially surrounded by a tube, the tube comprising a fluid impermeable material, the wick extending through the tube, the tube extending from the reservoir to the middle layer.

11. The moisture retaining mask of claim 10, the wick and tube at least partially disposed between the inner layer and outer layers.

12. The moisture retaining mask of claim 1, the reservoir is disposed beneath the main portion.

13. The moisture retaining mask of claim 12, the reservoir is disposed between the inner layer and outer layers.

14. The moisture retaining mask of claim 13, the wick extending from the reservoir to the middle layer between the inner and outer layers.

15. The moisture retaining mask of claim 1, further comprising an opening in the reservoir extending into the reservoir, the opening configured to support a tube, the tube and opening configured to convey a water containing fluid into the reservoir.

16. The moisture retaining mask of claim 15, the opening is disposed extending through the front layer.

17. The moisture retaining mask of claim 1, further comprising at least one strap, the at least one strap having a pair of strap ends, one of the strap ends attached to the left side of the main portion and the other of the strap ends attached to the right side of the main portion.

18. The moisture retaining mask of claim 1, the main portion configured to extend to substantially cover at least the mouth and nose of the user;

19. A method of adding water containing moisture to the breath of a user while the user is inhaling, comprising:
providing a moisture retaining mask comprising a main portion, the main portion having an outer layer, an inner layer, and a middle layer;
fitting the main portion to extend to substantially cover at least the mouth of the user;
providing the water containing moisture to a water absorbing material comprising the middle layer; and,
wherein the water containing moisture is provided by a wick extending from a reservoir holding water containing fluid to the middle layer, the reservoir disposed proximate the main portion.

20. A method of manufacturing a moisture retaining mask configured to be fitted over a mouth of a user to add water containing moisture to the breath of the user while the user is inhaling, comprising:
providing a main portion of the moisture retaining mask having a water resistant outer layer, a water resistant inner layer, and a water absorbing middle layer, the main portion configured to extend to substantially cover at least the mouth of the user;
providing the middle layer comprising a water absorbing material; and,
providing a wick configured to convey a water containing fluid, the wick provided extending from a reservoir holding the water containing fluid to the middle layer, the reservoir provided proximate the main portion.
